(19) United States
(12) Patent Application Publication
(10) Pub. No.: US 2022/0296935 A1
(43) Pub. Date: Sep. 22, 2022

(54) G TOLERANCE IMPROVEMENT DEVICE, G TOLERANCE IMPROVEMENT MASK AND G TOLERANCE IMPROVEMENT METHOD

(71) Applicant: NIPPON TELEGRAPH AND TELEPHONE CORPORATION, Tokyo (JP)

(72) Inventors: Shingo TSUKADA, Musashino-shi, Tokyo (JP); Hiroshi NAKASHIMA, Musashino-shi, Tokyo (JP); Toichiro GOTO, Musashino-shi,Tokyo (JP)

(73) Assignee: NIPPON TELEGRAPH AND TELEPHONE CORPORATION, Tokyo (JP)

(21) Appl. No.: 17/596,062

(22) PCT Filed: Jun. 4, 2019
(86) PCT No.: PCT/JP2019/022182

§ 371 Date: Dec. 2, 2021

(51) Int. Cl. A62B 7/14; A62B 18/02; B64D 10/00

(52) U.S. Cl. CPC A62B 7/14; A62B 18/02; B64D 10/00; B64D 2010/002

(57) ABSTRACT

An aspect of the present disclosure is a G-tolerance improving device including a valve control unit configured to control an operation of a main system valve configured to control pressure of oxygen to be supplied to a user who is breathing with positive pressure applied to his or her airway, the oxygen from a main supply source configured to supply the oxygen to the user, and an operation of an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source, in which, when a breathing state of the user changes from an exhalation phase to an inhalation phase, the valve control unit controls an operation of the main system valve and the auxiliary system valve such that the auxiliary gas is supplied to the user for a predetermined time that is shorter than a time of the inhalation phase.

### TECHNICAL FIELD

The present disclosure relates to a G-tolerance improving device, a G-tolerance improving mask, and a G-tolerance improving method.

### BACKGROUND ART

Traditionally, pilots of aircraft have suffered from abnormal conditions such as loss of vision, loss of consciousness, and disorder of the central nervous system when centrifugal forces are applied at the time of turning of the aircraft, and the like. These abnormal conditions (hereinafter referred to as “hypoxic brain conditions”) are caused by centrifugal forces exceeding an allowable amount resulting from turning of aircraft to reduce the venous return volume, which prevents a sufficient amount of oxygen from being fed to the brain. Further, in high-G environments, an imbalance in which the blood flow in the lungs is biased downward and ventilation is biased upward is caused, and thus hypoxia is more likely to occur. In addition, complex factors, such as a decrease in the fluid volume of the head and disturbance in the brain blood flow distribution, are also responsible for hypoxic brain conditions. Thus, in order to curb the occurrence of such hypoxic brain conditions and increase the tolerance of pilots to the centrifugal forces (hereinafter referred to as “G-tolerance”), the pilots perform hook breathing with wearing a mask that adjusts the airway pressure during flight. A mask that adjusts the airway pressure has a function of applying positive pressure to the airway pressure of pilots during exhalation and releasing the pressure during inhalation. Hook breathing is a way of breathing to repeat exhaling while closing the vocal cords and inhaling for a shorter time than the time of exhalation. In this way, the occurrence of the hypoxic brain conditions of pilots is curbed due to the mask that adjusts the airway pressure and hook breathing (see Non Patent Literatures 1 and 2).

### CITATION LIST

### Non Patent Literature

Non Patent Literature 1: James E. Whinnery, M. D., Ph. D. and Duane C. Murray, “Enhancing Tolerance to Acceleration (+Gz) Stress: The “Hook” Maneuver”, [online], [retrieved on May 22, 2019], Internet<URL: https://apps.dtic.mil/dtic/tr/fulltext/u2/a231094.pdf>

Non Patent Literature 2: “Aiming for Improvement in G-Tolerance of Pilots” Japanese Society of Pathophysiology, [online], [retrieved on May 22, 2019], Internet:<URL: http://byoutaiseiri.kenkyuukai.jp/images/sys %5Cinformation%5C20110304141052-81B83E59607896E5F806508E38B087CC28ECC3C9C242C4AE314ACAA1010C46FB.pdf>

### SUMMARY OF THE INVENTION

### Technical Problem

In such a method, pilots breathe under continuously applied positive pressure (positive pressure breathing). During breathing under continuous positive pressure, negative pressure is less likely to occur in the thoracic cavity, and as a result, venous return into the thoracic cavity is inhibited and cardiac output and cerebral blood flow decrease. Consequently, pilots may suffer hypoxic brain conditions. In addition, this problem is not limited to aircraft pilots, and is a common problem for racers and divers who perform labored breathing in high-G environments.

Taking the aforementioned circumstances into account, an objective of the present disclosure is to provide a technique for improving tolerance of a user to a strain on the body caused by acceleration.

### Means for Solving the Problem

An aspect of the present disclosure is a G-tolerance improving device including a valve control unit configured to control an operation of a main system valve configured to control pressure of oxygen to be supplied to a user who is breathing with positive pressure applied to his or her airway, the oxygen from a main supply source configured to supply the oxygen to the user, and an operation of an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source, in which, when a breathing state of the user changes from an exhalation phase to an inhalation phase, the valve control unit controls an operation of the main system valve and the auxiliary system valve such that the auxiliary gas is supplied to the user for a predetermined time that is shorter than a time of the inhalation phase.

An aspect of the present disclosure is the G-tolerance improving device, in which pressure of the oxygen supplied from the main supply source in the inhalation phase is lower than pressure of the oxygen supplied from the main supply source in the exhalation phase.

An aspect of the present disclosure is the G-tolerance improving device further including a pressure sensor configured to measure an airway pressure of the user, in which the valve control unit controls an operation of the main system valve and the auxiliary system valve based on a measurement result of the pressure sensor.

An aspect of the present disclosure is the G-tolerance improving device further including a breathing state determination unit configured to determine a breathing state of the user based on a measurement result of the pressure sensor, in which the valve control unit controls an operation of the main system valve and the auxiliary system valve based on a determination result of the breathing state determination unit.

An aspect of the present disclosure is the G-tolerance improving device further including a hyperventilation determination unit configured to determine whether the user is in a hyperventilation state, in which the auxiliary gas includes the highly concentrated oxygen and carbon dioxide, and when the hyperventilation determination unit determines that the user is in a hyperventilation state, the valve control unit controls an operation of the main system valve and the auxiliary system valve such that the auxiliary gas is supplied to the user for a predetermined time that is shorter than the time of the inhalation phase.

An aspect of the present disclosure is a G-tolerance improving mask including a mask body configured to cover a mouth of a user who is breathing with positive pressure applied to his or her airway, a pressure sensor configured to measure a pressure inside the mask body, a main system valve configured to control pressure of oxygen to be supplied to the user from a main supply source configured to supply oxygen to the user, and an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source, in which the main system valve and the auxiliary system valve operate based on a measurement result of the pressure sensor such that, when a breathing state of the user changes from an exhalation phase to an inhalation phase, the auxiliary gas is supplied to the user for a predetermined time that is shorter than a time of the inhalation phase.

An aspect of the present disclosure is a G-tolerance improving method performed by a G-tolerance improving device including a valve control unit configured to control an operation of a main system valve configured to control pressure of oxygen to be supplied to a user who is breathing with positive pressure applied to his or her airway, the oxygen from a main supply source configured to supply the oxygen to the user, and an operation of an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source, the G-tolerance improving method including controlling, by the valve control unit, when a breathing state of the user changes from an exhalation phase to an inhalation phase, an operation of the main system valve and the auxiliary system valve such that the auxiliary gas is supplied to the user for a predetermined time that is shorter than a time of the inhalation phase.

### EFFECTS OF THE INVENTION

The present disclosure enables a user to improve tolerance to a load on the body caused by acceleration.

### BRIEF DESCRIPTION OF DRAWINGS

FIG. 1 is a diagram illustrating a usage example of a G-tolerance improving device 1 according to a first embodiment.

FIG. 2 is a diagram illustrating an example of a functional configuration of a control unit 41 according to the first embodiment.

FIG. 3 is a flowchart showing an example of oxygen supply control processing according to the first embodiment.

FIG. 4 is a diagram illustrating an example of experimental results of changes in airway pressure of a user 91 and changes in pressure of gases supplied by a main cylinder 11 and an auxiliary cylinder 12 according to the first embodiment.

FIG. 5 is a diagram illustrating a usage example of a G-tolerance improving device la according to a second embodiment.

FIG. 6 is a diagram illustrating an example of a functional configuration of a control unit 41a according to the second embodiment.

FIG. 7 is a flowchart showing an example of hyperventilation-time valve control processing according to the second embodiment.

### DESCRIPTION OF EMBODIMENT

### First Embodiment

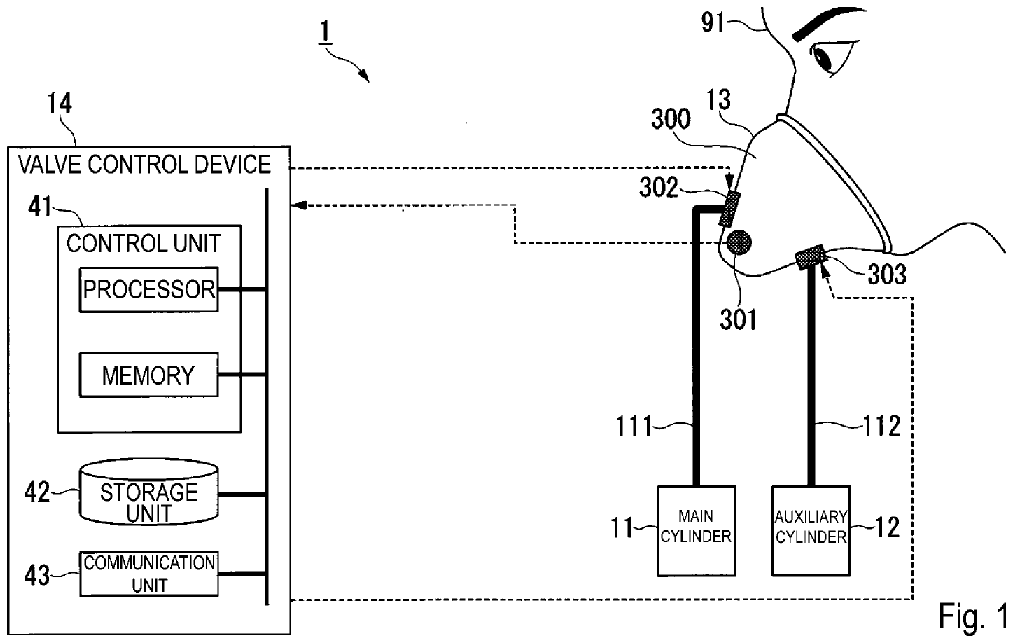

FIG. 1 is a diagram illustrating a usage example of a G-tolerance improving device 1 according to a first embodiment. A user 91 breathes with positive pressure continuously supplied from a mask to the airway. In other words, the user 91 performs pressurized breathing with the mask. The user 91 also performs hook breathing in pressurized breathing. Hook breathing is breathing in which the labored exhalation phase is extended with the vocal cords closed, and the inhalation phase is shortened while airway pressure is maintained at positive pressure for a long time. That is, hook breathing is breathing in a state in which positive pressure is continuously applied for a long time. In hook breathing, the user 91 continuously exhales with the vocal cords closed under positive pressure. “Under positive pressure” refers to a state of the user 91 to whom oxygen is being supplied. The time of the inhalation phase is shorter than the time of the exhalation phase in hook breathing. The time from the start of an exhalation phase to the start of the next exhalation phase after going through an inhalation phase is, for example, about 3 seconds. In the case of exhalation under positive pressure, the venous return to the thoracic cavity is inhibited.

The G-tolerance improving device 1 releases a main gas at a first pressure when the user 91 exhales. Specifically, the main gas is oxygen. The G-tolerance improving device 1 releases an auxiliary gas at a second pressure for a predetermined period of time (hereinafter referred to as a “bolus time”) when the user 91 inhales. The auxiliary gas may be a gas having a higher concentration than the main gas or may be a different gas. The auxiliary gas may be, for example, oxygen having a higher concentration than the main gas. The auxiliary gas may be, for example, a mixed gas of highly concentrated oxygen and carbon dioxide. The G-tolerance improving device 1 releases the main gas at a third pressure that is lower than the first pressure and the second pressure in a time other than the bolus time during inhalation. The G-tolerance improving device 1 releases the auxiliary gas at a fourth pressure that is lower than the first pressure, the second pressure, and the third pressure in a time other than the bolus time during inhalation. The fourth pressure is a pressure of about 0. A case in which the main gas is oxygen and the auxiliary gas is highly concentrated oxygen will be described below as an example.

The G-tolerance improving device 1 includes a main cylinder 11, an auxiliary cylinder 12, a G-tolerance improving mask 13, and a valve control device 14. The main cylinder 11 supplies the main gas to the G-tolerance improving mask 13 via a main system tube 111. The auxiliary cylinder 12 supplies the auxiliary gas to the G-tolerance improving mask 13 via an auxiliary system tube 112. The main system tube 111 is a hollow tube in which the main gas flows. The auxiliary system tube 112 is a hollow tube in which the auxiliary gas flows.

The G-tolerance improving mask 13 includes a mask body 300, a pressure sensor 301, a main system valve 302, and an auxiliary system valve 303. The mask body 300 covers the mouth of the user 91. The pressure sensor 301 measures airway pressure of the user 91. For example, the pressure sensor 301 measures pressure inside the mask body 300 as airway pressure. The pressure sensor 301 may be positioned anywhere along the path connecting to the airway including a location on the mask body 300, on the main system tube 111, on a connector connecting the main system tube 111 to the mask body 300, and the like. The G-tolerance improving mask 13 is connected to the main system tube 111 via the main system valve 302. The main gas that has flowed through the main system tube 111 flows into the mask body 300. The G-tolerance improving mask 13 is connected to the auxiliary system tube 112 via the auxiliary system valve 303. The auxiliary gas that has flowed through the auxiliary system tube 112 flows into the mask body 300.

The main system valve 302 operates under control of a valve control device 14. The main system valve 302 operates under control of the valve control device 14 to control pressure of the main gas supplied to the G-tolerance improving mask 13. The auxiliary system valve 303 operates under control of a valve control device 14. The auxiliary system valve 303 operates under control of the valve control device 14 to control pressure of the auxiliary gas supplied to the G-tolerance improving mask 13.

The valve control device 14 controls the operations of the main system valve 302 and the auxiliary system valve 303 in accordance with measurement results of the pressure sensor 301. The valve control device 14 includes a control unit 41 including a processor such as a central processing unit (CPU) and a memory connected to each other by a bus and executes a program. The valve control device 14 functions as a device including the control unit 41, a storage unit 42, a communication unit 43, and an input unit 44 by executing the program.

The control unit 41 controls operations of each of the functional units of the valve control device 14. The control unit 41 controls operations of the main system valve 302 and the auxiliary system valve 303 in accordance with measurement results of the pressure sensor 301. The control unit 41 acquires the measurement results of the pressure sensor 301 via the communication unit 43. The control unit 41 controls the operations of the main system valve 302 and the auxiliary system valve 303 by transmitting control signals to the main system valve 302 and the auxiliary system valve 303 via the communication unit 43.

The storage unit 42 is configured using a storage device such as a magnetic hard disk device or a semiconductor storage device. The storage unit 42 stores various types of information related to the valve control device 14. The storage unit 42 stores the measurement results of the pressure sensor 301, for example. The storage unit 42 stores a history of control of the main system valve 302 and the auxiliary system valve 303, for example.

The communication unit 43 is configured to include a communication interface for wireless or wired communication between the valve control device 14 and the pressure sensor 301. The communication unit 43 is configured to include a communication interface for wireless or wired communication between the valve control device 14 and the main system valve 302. The communication unit 43 is configured to include a communication interface for wireless or wired communication between the valve control device 14 and the auxiliary system valve 303.

The input unit 44 is configured to include an input device such as a touch panel. The input unit 44 may be configured as an interface for connecting the input device to the own valve control device. The input unit 44 receives inputs of a start instruction and an end instruction for the own valve control device. The start instruction indicates a start of control of the main system valve 302 and the auxiliary system valve 303 by the control unit 41. The end instruction indicates an end of control of the main system valve 302 and the auxiliary system valve 303 by the control unit 41. The input unit 44 outputs the input start instruction and end instruction to the control unit 41. The control unit 41 that has acquired the start instruction starts control of the main system valve 302 and the auxiliary system valve 303. The control unit 41 that has acquired the end instruction ends control of the main system valve 302 and the auxiliary system valve 303.

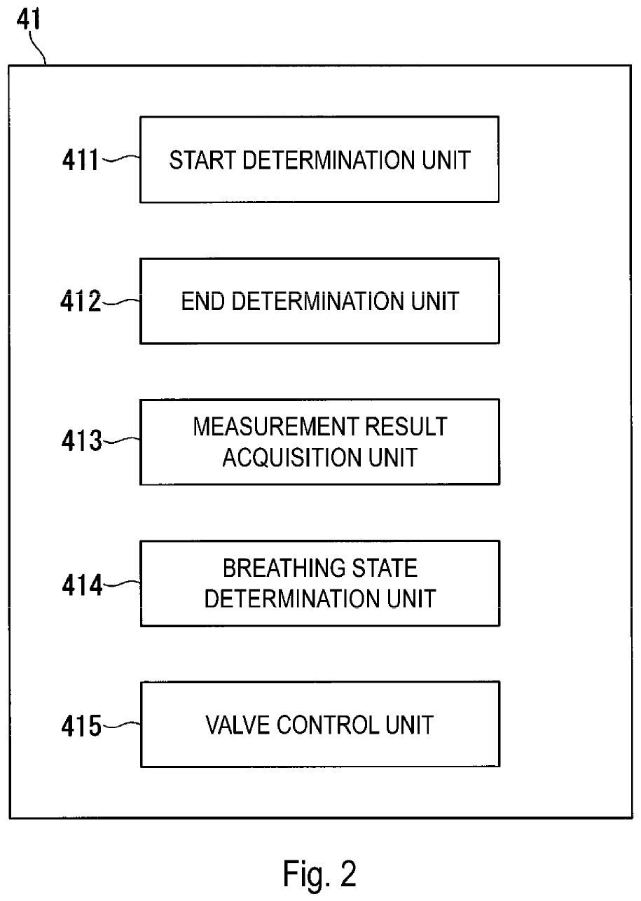

FIG. 2 is a diagram illustrating an example of a functional configuration of the control unit 41 according to the first embodiment. The control unit 41 includes a start determination unit 411, an end determination unit 412, a measurement result acquisition unit 413, a breathing state determination unit 414, and a valve control unit 415.

The start determination unit 411 determines whether there is a start instruction input to the input unit 44. When the start determination unit 411 determines that there is an input of a start instruction, the start determination unit 411 starts operations of the measurement result acquisition unit 413, the breathing state determination unit 414, and the valve control unit 415. The end determination unit 412 determines whether there is an end instruction input to the input unit 44. When the end determination unit 412 determines that there is an input of an end instruction, the end determination unit 412 ends operations of the measurement result acquisition unit 413, the breathing state determination unit 414, and the valve control unit 415. Hereinafter, processing executed by the G-tolerance improving device 1 from when a start instruction is input to the input unit 44 to when an end instruction is input to the input unit 44 is referred to as oxygen supply control processing. The measurement result acquisition unit 413 acquires a measurement result of the pressure sensor 301 (hereinafter referred to as a “pressure measurement result”).

The breathing state determination unit 414 determines whether a condition indicating that a state of breathing of the user 91 changed from an exhalation phase to an inhalation phase (hereinafter referred to as a “first breathing state condition”) has been satisfied based on a pressure measurement result. The first breathing state condition may be a condition that, for example, the airway pressure drops by a predetermined pressure after being at a first airway pressure for a first predetermined time. The first airway pressure is positive. The breathing state determination unit 414 determines whether a condition related to a state of breathing of the user 91 and a change from an inhalation phase to an exhalation phase (hereinafter referred to as a “second breathing state condition”) has been satisfied based on a pressure measurement result. The second breathing state condition may be a condition that, for example, the airway pressure increases by a predetermined pressure after being at a second airway pressure for a second predetermined time. The second airway pressure is negative. The second breathing state condition may be a condition that, for example, the second predetermined time elapses with the airway pressure at the second airway pressure. Hereinafter, a case in which the second breathing state condition is the condition that the second predetermined time elapses with the airway pressure at the second airway pressure will be described as an example. Further, positive pressure and negative pressure in the present description do not necessarily have the reference of a ground-based pressure (one barometric pressure) at 0, and may have a relative reference in accordance with the environment. For example, positive pressure and negative pressure may be roughly set based on an internal pressure of a cabin, an average pressure of a breathing device, or the like.

When the start determination unit 411 determines that a start instruction has been input to the input unit 44, the valve control unit 415 executes the first pressure control. The first pressure control is control in which the operations of the main system valve 302 and the auxiliary system valve 303 are controlled such that pressure of the main gas supplied by the main cylinder 11 is controlled at the first pressure and pressure of the auxiliary gas supplied by the auxiliary cylinder 12 is controlled at the fourth pressure. The valve control unit 415 executes the first pressure control until the first breathing state condition is satisfied.

The valve control unit 415 executes second pressure control when the first breathing state condition is satisfied. The second pressure control is control in which the operations of the main system valve 302 and the auxiliary system valve 303 are controlled such that pressure of the main gas supplied by the main cylinder 11 is controlled at the third pressure and pressure of the auxiliary gas supplied by the auxiliary cylinder 12 is controlled at the second pressure. The second pressure control is control performed for a bolus time.

The valve control unit 415 executes third pressure control until the second breathing state condition is satisfied after the execution of the second pressure control. The third pressure control is control in which the operations of the main system valve 302 and the auxiliary system valve 303 are controlled such that pressure of the main gas supplied by the main cylinder 11 is controlled at the third pressure and pressure of the auxiliary gas supplied by the auxiliary cylinder 12 is controlled at the fourth pressure. The valve control unit 415 executes the first pressure control when the second breathing state condition is satisfied. In this way, the valve control unit 415 controls the operations of the main system valve 302 and the auxiliary system valve 303 based on the determination results of the breathing state determination unit 414.

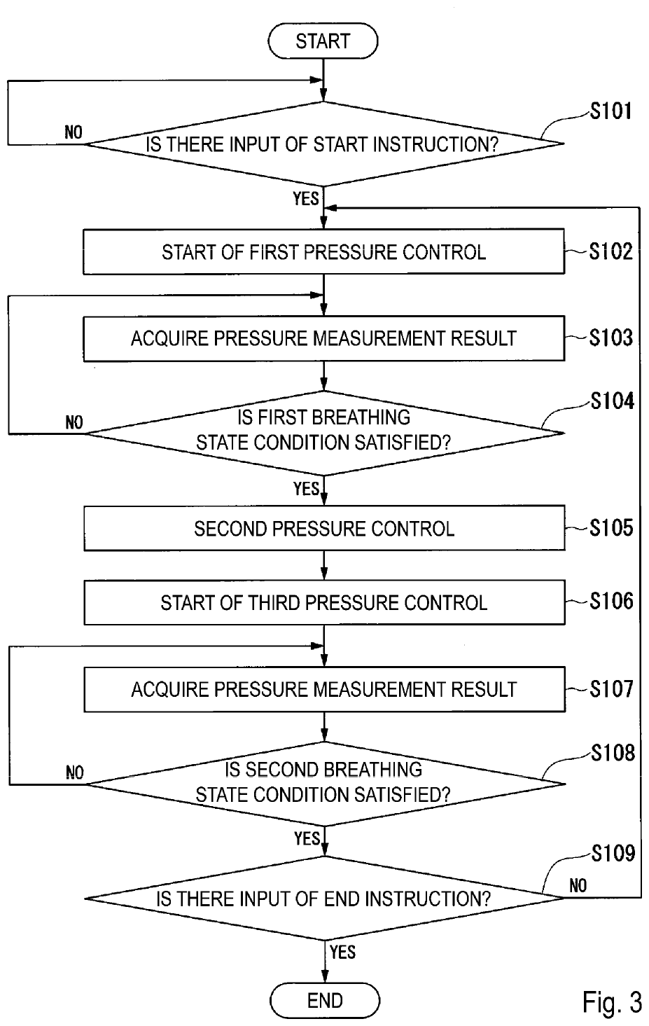

FIG. 3 is a flowchart showing an example of oxygen supply control processing according to the first embodiment.

The start determination unit 411 determines whether there is a start instruction input to the input unit 44 (step S101). If there is no start instruction input to the input unit 44 (step S101: NO), the process returns to step S101. On the other hand, if there is a start instruction input to the input unit 44 (step S101: YES), the valve control unit 415 starts the first pressure control (step S102). The measurement result acquisition unit 413 acquires pressure measurement results (step S103). The breathing state determination unit 414 determines whether the first breathing state condition has been satisfied based on the pressure measurement results (step S104). If the first breathing state condition is not satisfied (step S104: NO), the process returns to step S103. Further, the first pressure control is continuously executed during the execution of step S103 and step S104.

If the first breathing state condition is satisfied (step S104: YES), the valve control unit 415 executes the second pressure control for a bolus time (step S105). Next to step S105, the valve control unit 415 starts executing the third pressure control (step S106). The measurement result acquisition unit 413 acquires the pressure measurement results (step S107). The breathing state determination unit 414 determines whether the second breathing state condition has been satisfied based on the pressure measurement results (step S108). If the second breathing state condition is not satisfied (step S108: NO), the process returns to step 5107. Further, the second pressure control is continuously executed during the execution of step S107 and step S108.

If the second breathing state condition is satisfied (step S108: YES), the end determination unit 412 determines whether there is an end instruction input to the input unit 44 (step S109). If there is no end instruction input to the input unit 44 (step S109: NO), the process returns to step S102. In other words, execution of the first pressure control is started. If there is an end instruction input to the input unit 44 (step S109: YES), the oxygen supply control processing ends. Further, the processing of step S109 need not be executed next to the processing of step S108 at all times, and may be executed at any timing after step S101.

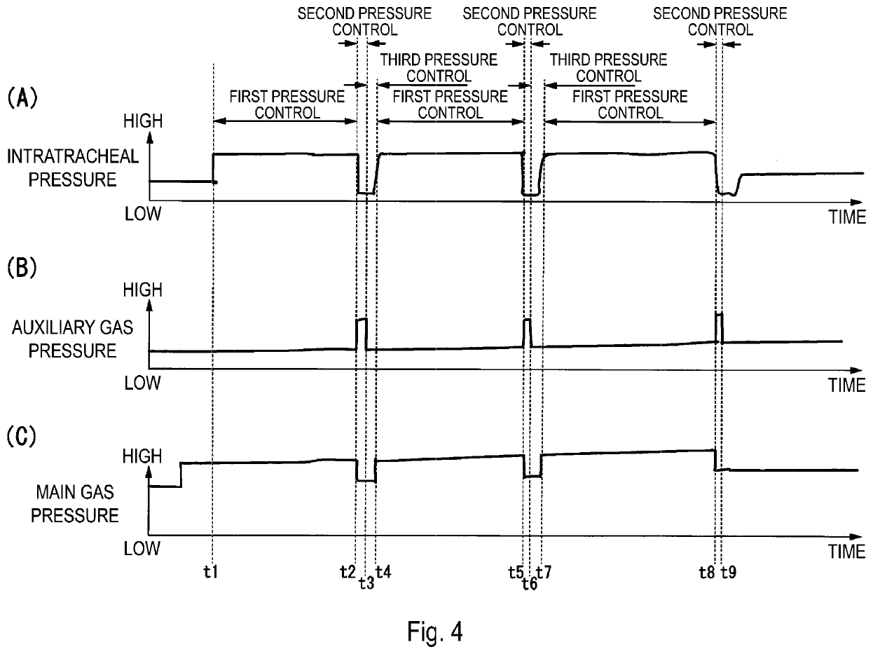

FIG. 4 is a diagram illustrating an example of experimental results of changes in airway pressure of the user 91 using the G-tolerance improving device 1 of the first embodiment and changes in pressure of gases supplied by the main cylinder 11 and the auxiliary cylinder 12. In FIG. 4, the breathing state of the user 91 represents a breathing state of the user 91 performing hook breathing.

FIG. 4(A) shows changes over time in airway pressure of a user 91 performing hook breathing. The vertical axis of the graph in FIG. 4(A) represents airway pressure. The horizontal axis of the graph in FIG. 4(A) represents time. The vertical axis of the graph in FIG. 4(B) represents pressure of the auxiliary gas. The horizontal axis of the graph in FIG. 4(B) represents time. The vertical axis of the graph in FIG. 4(C) represents pressure of the main gas. The horizontal axis of the graph in FIG. 4(C) represents time.

FIG. 4 shows that the airway pressure of the user 91 increased at a time t1. In FIG. 4, the breathing state of the user 91 from the time t1 to a time t2 is an exhalation phase. That is, the user 91 continuously exhaled with the vocal cords closed from the time t1 to the time t2. The airway pressure from the time t1 to the time t2 is positive. FIG. 4 shows that the first pressure control was executed from the time t1 to the time t2.

FIG. 4 shows that the airway pressure of the user 91 decreased at the time t2. FIG. 4 shows that the first breathing state condition was satisfied and the second pressure control was started at the time t2. FIG. 4 shows that the second pressure control was executed from the time t2 to a time t3. The time from the time t2 to the time t3 is an example of a bolus time. FIG. 4 shows that the third pressure control was executed from the time t3 to a time t4. FIG. 4 shows that the airway pressure of the user 91 increased at the time t4. FIG. 4 shows that the second breathing state condition was satisfied and the first pressure control was started at the time t4. In FIG. 4, the breathing state of the user 91 from the time t2 to the time t4 is an inhalation phase. That is, the user 91 inhaled from the time t2 to the time t4. The airway pressure from the time t2 to the time t4 is negative.

In FIG. 4, the breathing state of the user 91 from the time t4 to a time t5 is an exhalation phase. That is, the user 91 continuously exhaled with the vocal cords closed from the time t4 to the time t5. FIG. 4 shows that the first pressure control was executed from the time t4 to the time t5. The airway pressure from the time t4 to the time t5 is positive.

FIG. 4 shows that the airway pressure of the user 91 decreased at the time t5. FIG. 4 shows that the first breathing state condition was satisfied and the second pressure control was started at the time t5. FIG. 4 shows that the second pressure control was executed from the time t5 to a time t6. The time from the time t5 to the time t6 is an example of a bolus time. FIG. 4 shows that the third pressure control was executed from the time t6 to a time t7. FIG. 4 shows that the airway pressure of the user 91 increased at the time t7. FIG. 4 shows that the second breathing state condition was satisfied and the first pressure control was started at the time t7. In FIG. 4, the breathing state of the user 91 from the time t5 to the time t7 is an inhalation phase. That is, the user 91 inhaled from the time t5 to the time t7. The airway pressure from the time t5 to the time t7 is negative.

In FIG. 4, the breathing state of the user 91 from the time t7 to a time t8 is an exhalation phase. That is, the user 91 continuously exhaled with the vocal cords closed from the time t7 to the time t8. FIG. 4 shows that the first pressure control was executed from the time t7 to the time t8. FIG. 4 shows that the airway pressure of the user 91 decreased at the time t8. FIG. 4 shows that the first breathing state condition was satisfied and the second pressure control was started at the time t8. FIG. 4 shows that the second pressure control was executed from the time t8 to a time t9. The time from the time t8 to the time t9 is an example of a bolus time. The airway pressure from the time t7 to the time t8 is positive and the airway pressure from the time t8 to the time t9 is negative.

The G-tolerance improving device 1 according to the first embodiment configured as described above supplies oxygen at a higher concentration in the inhalation phases in breathing of the user 91 performing hook breathing than oxygen supplied in the exhalation phase for the bolus times. Thus, the G-tolerance improving device 1 can supply oxygen at a high concentration to the user 91 with no inhibited venous return to the thoracic cavity. Therefore, the G-tolerance improving device 1 can curb a decrease in a cerebral blood flow caused by inhibition of venous return to the thoracic cavity of the user 91 performing hook breathing, and can improve tolerance of the user 91 to the load on the body imposed due to acceleration.

### Second Embodiment

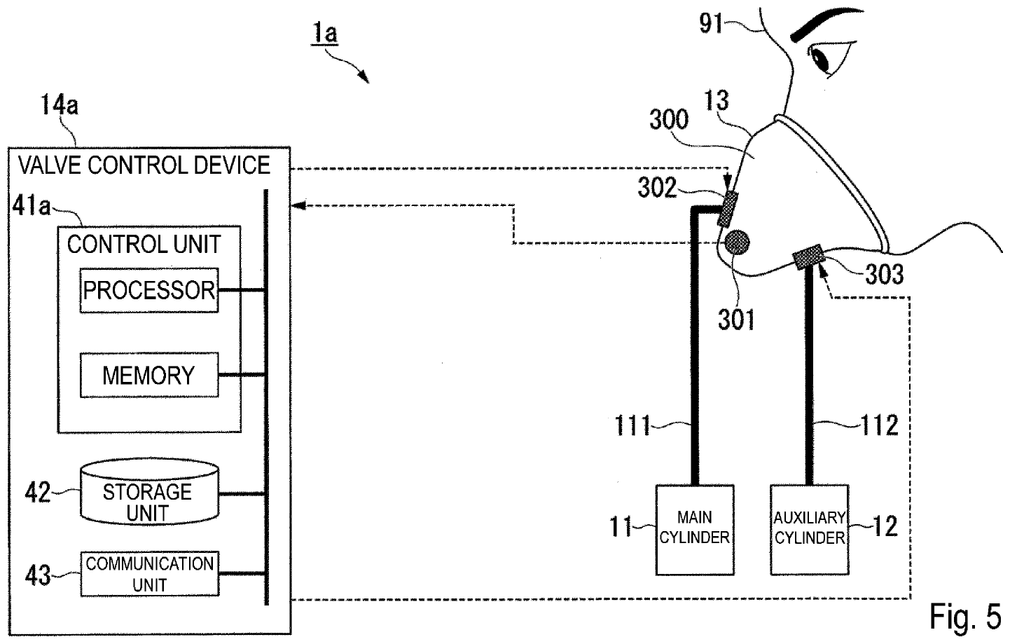

FIG. 5 is a diagram illustrating a usage example of a G-tolerance improving device la according to a second embodiment. The G-tolerance improving device 1a differs from the G-tolerance improving device 1 in that a valve control device 14a is provided in place of the valve control device 14. The valve control device 14a differs from the valve control device 14 in that a control unit 41a is provided in place of the control unit 41. The control unit 41a includes a processor such as a CPU and a memory. An auxiliary cylinder 12 included in the G-tolerance improving device 1a is a mixed gas of highly concentrated oxygen and carbon dioxide.

Hereinafter, the same reference numerals as those in FIG. 1 are given to functional units having the same functions as those provided in the G-tolerance improving device 1, and description thereof is be omitted.

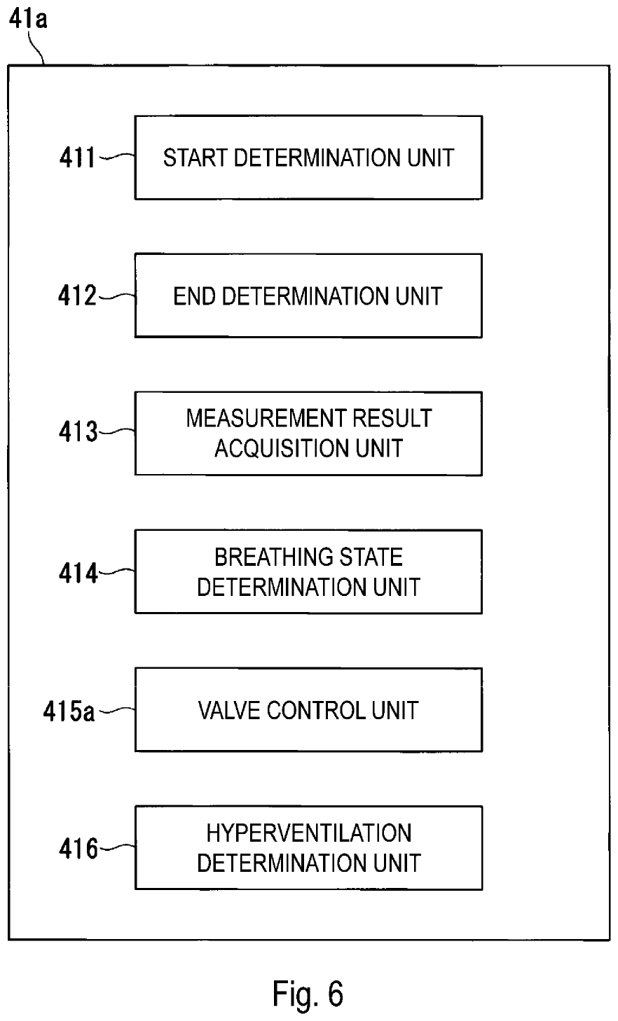

FIG. 6 is a diagram illustrating an example of a functional configuration of the control unit 41a according to the second embodiment. The control unit 41a differs from the control unit 41 in that a hyperventilation determination unit 416 is provided, and a valve control unit 415a is provided instead of the valve control unit 415. Hereinafter, the same reference numerals as those in FIG. 2 are given to functional units having the same functions as those provided in the control unit 41, and description thereof is be omitted.

The hyperventilation determination unit 416 determines whether the user 91 is in a hyperventilation state based on pressure measurement results. The hyperventilation determination unit 416 may identify why the user 91 is in a hyperventilation state if it can determine that the user 91 is in a hyperventilation state based on the pressure measurement results. For example, the hyperventilation determination unit 416 determines that the user 91 has hyperventilation when the pressure measurement results indicate that a change in positive pressure and negative pressure has occurred a predetermined number of times or more within a predetermined time.

The valve control unit 415a executes hyperventilation-time valve control processing in addition to the processing executed by the valve control unit 415 according to the first embodiment. The hyperventilation-time valve control processing is processing to perform the second pressure control in a case where the hyperventilation determination unit 416 determines that the user 91 has hyperventilation. The processing executed by the valve control unit 415 according to the first embodiment includes the oxygen supply control processing illustrated in the flowchart of FIG. 3.

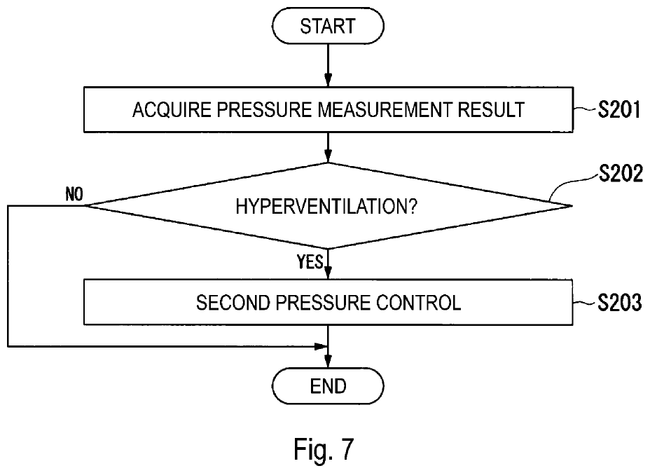

FIG. 7 is a flowchart showing an example of hyperventilation-time valve control processing according to the second embodiment. The hyperventilation-time valve control processing is repeated. A measurement result acquisition unit 413 acquires pressure measurement results (step S201). The hyperventilation determination unit 416 determines whether the user 91 is in a hyperventilation state based on pressure measurement results (step S202). If the user 91 is determined to be in a hyperventilation state (step S202: YES), the valve control unit 415a executes the second pressure control for a bolus time (step S203). On the other hand, if the user 91 is determined to be not in a hyperventilation state (step S202: NO), the hyperventilation-time valve control processing ends.

The G-tolerance improving device la of the second embodiment configured as described above determines whether the user 91 is in a hyperventilation state, and supplies carbon dioxide when the breathing of the user 91 is in an inhalation phase if the user 91 has hyperventilation. Therefore, the G-tolerance improving device 1a can help the user 91 in a hyperventilation state shift to a state without hyperventilation.

### Modified Example

A medical amount of auxiliary gas that is desirable to be supplied to the user 91 in one time of the second breathing control has been determined. In addition, a medically desirable concentration of the auxiliary gas is determined as well. Thus, there is a desirable time for a bolus time, and a bolus time is desirably within 1 second. It is particularly desirable that a bolus time be substantially 0.3 seconds.

Further, the main cylinder 11 is an example of a main supply source. Further, the auxiliary cylinder 12 is an example of an auxiliary supply source.

Further, all or some functions of the G-tolerance improving device 1 may be realized using hardware such as an application specific integrated circuit (ASIC), a programmable logic device (PLD), or a field programmable gate array (FPGA). A program may be recorded in a computer-readable recording medium. The computer-readable recording medium is, for example, a portable medium such as a flexible disk, a magneto-optical disk, a read only memory (ROM), or a compact disk read only memory (CD-ROM), or a storage device such as a hard disk built in a computer system. The program may be transmitted via an electrical communication line.

Although the embodiments of the present disclosure have been described above in detail with reference to the drawings, specific configurations are not limited to those embodiments, and any design or the like within the scope not departing from the gist of the present disclosure is also included.

### REFERENCE SIGNS LIST

1 G-tolerance improving device

11 Main cylinder

12 Auxiliary cylinder

13 G-tolerance improving mask

14, 14a Valve control device

41, 41a Control unit

42 Storage unit

43 Communication unit

111 Main system tube

112 Auxiliary system tube

300 Mask body

301 Pressure sensor

302 Main system valve

303 Auxiliary system valve

411 Start determination unit

412 End determination unit

413 Measurement result acquisition unit

414 Breathing state determination unit

415, 415a Valve control unit

416 Hyperventilation determination unit

1. A G-tolerance improving device comprising
a valve control unit configured to control an operation of a main system valve configured to control pressure of oxygen to be supplied to a user who is breathing with positive pressure applied to his or her airway, the oxygen from a main supply source configured to supply the oxygen to the user, and an operation of an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source,
wherein, when a breathing state of the user changes from an exhalation phase to an inhalation phase, the valve control unit controls an operation of the main system valve and the auxiliary system valve such that the auxiliary gas is supplied to the user for a predetermined time that is shorter than a time of the inhalation phase.

2. The G-tolerance improving device according to claim 1,
wherein pressure of the oxygen supplied from the main supply source in the inhalation phase is lower than pressure of the oxygen supplied from the main supply source in the exhalation phase.

3. The G-tolerance improving device according to claim 1 or 2, further comprising
a pressure sensor configured to measure an airway pressure of the user,
wherein the valve control unit controls an operation of the main system valve and the auxiliary system valve based on a measurement result of the pressure sensor.

4. The G-tolerance improving device according to claim 3, further comprising
a breathing state determination unit configured to determine a breathing state of the user based on a measurement result of the pressure sensor,
wherein the valve control unit controls an operation of the main system valve and the auxiliary system valve based on a determination result of the breathing state determination unit.

5. The G-tolerance improving device according to claim 1, further comprising
a hyperventilation determination unit configured to determine whether the user is in a hyperventilation state,
wherein the auxiliary gas includes the highly concentrated oxygen and carbon dioxide, and when the hyperventilation determination unit determines that the user is in a hyperventilation state, the valve control unit controls an operation of the main system valve and the auxiliary system valve such that the auxiliary gas is supplied to the user for a predetermined time that is shorter than the time of the inhalation phase.

6. A G-tolerance improving mask comprising:
a mask body configured to cover a mouth of a user who is breathing with positive pressure applied to his or her airway,
a pressure sensor configured to measure a pressure inside the mask body,
a main system valve configured to control pressure of oxygen to be supplied to the user from a main supply source configured to supply oxygen to the user, and
an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source,
wherein the main system valve and the auxiliary system valve operate based on a measurement result of the pressure sensor such that, when a breathing state of the user changes from an exhalation phase to an inhalation phase, the auxiliary gas is supplied to the user for a predetermined time that is shorter than a time of the inhalation phase.

7. A G-tolerance improving method performed by a G-tolerance improving device including a valve control unit configured to control an operation of a main system valve configured to control pressure of oxygen to be supplied to a user who is breathing with positive pressure applied to his or her airway, the oxygen from a main supply source configured to supply the oxygen to the user, and an operation of an auxiliary system valve configured to control pressure of an auxiliary gas to be supplied to the user, the auxiliary gas from an auxiliary supply source configured to supply, to the user, the auxiliary gas including highly concentrated oxygen that is oxygen at a higher concentration than the oxygen supplied from the main supply source, the G-tolerance improving method comprising
controlling, by the valve control unit, when a breathing state of the user changes from an exhalation phase to an inhalation phase, an operation of the main system valve and the auxiliary system valve such that the auxiliary gas is supplied to the user for a predetermined time that is shorter than a time of the inhalation phase.
